(19) United States
(12) Patent Application Publication
(10) Pub. No.: US 2024/0248032 A1
(43) Pub. Date: Jul. 25, 2024

(54) LUNG CANCER DIAGNOSTIC

(71) Applicant: Vindico ICS Ltd., Llanelli (GB)

(72) Inventors: Paul Lewis, Llanelli (GB); Charles Brilliant, Swansea (GB)

(73) Assignee: Vindico ICS Ltd., Llanelli (GB)

(21) Appl. No.: 18/251,373

(22) PCT Filed: Oct. 28, 2021
(86) PCT No.: PCT/EP2021/080042

§ 371 Date: May 1, 2023

(30) Foreign Application Priority Data
Nov. 2, 2020 (GB) 2017340.7

(51) Int. Cl. G01N 21/3577; G01N 1/30; G01N 21/35

(52) U.S. Cl. CPC G01N 21/3577; G01N 1/30; G01N 2021/3595

(57) ABSTRACT

The invention concerns a method for determining lung cancer in a human subject with a high risk or likelihood of developing lung cancer comprising performing FTIR spectral analysis of a sputum sample obtained from the subject and, optionally, comparing the spectrum from the sample with that of a control; use of said method to further select a course of treatment for lung cancer and a method of treatment comprising same; and a kit of parts for use in said method.

### CROSS-REFERENCE TO RELATED APPLICATIONS

This is the U.S. National Stage of International Application No. PCT/EP2021/080042, filed Oct. 28, 2021, which was published in English under PCT Article 21(2), which claims the benefit of GB Application No. 2017340.7 filed Nov. 2, 2020. The PCT application is herein incorporated by reference in its entirety.

### FIELD OF THE INVENTION

The invention concerns a method for determining lung cancer in a human subject with a high risk or likelihood of developing lung cancer, said method comprising performing FTIR spectral analysis of a sputum sample obtained from the subject and, optionally, comparing a portion of the spectrum from the sample with that of a control; use of said method to further select a course of treatment for lung cancer and a method of treatment comprising same; and a kit of parts for use in said method.

### BACKGROUND OF THE INVENTION

Worldwide, lung cancer represents a huge burden on healthcare systems and is a major cause of mortality. It is the most common cause of cancer in adult males (16.7%) and the third most common in adult females (8.7%) and is the responsible for 23.6% and 13.6% of all cancer deaths in adult males and adult females, respectively. Lung cancer patients also have a very poor 5-year survival rate of <10%, which is primarily due to a majority of patients being diagnosed only after the disease has progressed to a stage that no longer can be easily treated.

The currently available methods for detection of lung cancer include flexible bronchoscopy, computed tomography (CT)-scan and X-ray. However, these techniques are not effective for early detection of the disease, as evidenced by the extremely poor rate of diagnosis of early stage disease. Flexible bronchoscopy has been shown to have an overall sensitivity for lung cancer diagnosis of 88%, however this sensitivity drops markedly for peripheral lesions of <2 cm in diameter to 34%. Almost 1 in 4 (23%) of diagnostic X-rays have been shown to provide a false negative results, whilst CT-scan has been shown to have 88.9% sensitivity and 92.6% specificity for diagnosis of lung cancer in a study comparing X-ray to CT-scanning. However, CT-scanning is limited by the potential for over-diagnosis and causing radiation-related harm to the patient. Evidently, there is a clear unmet need for a highly sensitive and specific diagnostic tool, capable of diagnosing both centrally located and peripherally located lesions, whilst causing minimal harm and distress to the patient.

Fourier transform infrared (FTIR) spectroscopy is a highly-sensitive analytical method which is capable of rapidly analysing structural changes in molecules. Due to its inherent ease of use, high reproducibility and non-invasiveness, FTIR has previously been applied with success to a range of biofluids and tissue samples. The technique is capable of analysing microlitre volumes of sample, with minimal sample preparation required. FTIR has shown promise as a sensitive diagnostic tool to distinguish neoplastic from normal cells in cancers such as colon cancer, prostate, breast, cervical, gastric, oral and oesophageal cancer. Briefly, FTIR measures chemical bond vibrations by measuring infrared (IR) absorbance by a sample—or transmission through a sample—and then produces an infrared spectrum based on the absorptive or transmissive properties of that sample. Depending on the analysis, changes in IR spectra, including readings at specific wavenumber or wavenumber regions, can be used to infer changes in sample composition that may correlate with a disease condition.

Indeed, it has previously been shown that FTIR can be used as a method for identifying biochemical changes in processed pelleted sputum as biomarkers for detection of lung cancer. Sputum was collected from lung cancer patients and healthy controls who showed no previous history of cancer or lung disease. FTIR spectra were generated from sputum cell pellets using infrared wavenumbers within the 1800 to 950 cm⁻¹ “fingerprint” region, identifying certain regions of importance in diagnosis.

This study was limited in that the diagnostic power of the identified regions could differentiate lung cancer from non-cancer with only 80% sensitivity and specificity, and only when comparing to healthy individuals. Further, and more significantly, there are many major contributors to developing lung cancer, including important environmental and occupational risks, as well a multitude of genetic factors, which can skew diagnosis testing. Patients of chronic obstructive pulmonary disease (COPD), a common smoking-related obstructive respiratory disease, have a higher risk of developing lung cancer as their forced expiratory volume in one second (FEV1) declines. Diagnosis of lung cancer in such individuals is influenced by the patients' background with a late stage diagnosis more likely in presence of comorbidities and disability, with COPD as a comorbid condition being strongly associated with stage-independent poor survival. Indeed, some COPD patients, especially those who have frequent exacerbations, can be accustomed to frequent changes in their condition including their tissue samples of relevance for diagnosis (i.e. sputum in this case), and this may contribute further to a late-stage diagnosis of cancer as persistent changes to symptoms could be attributed to an exacerbation. This can thus lead to erroneous or incorrect diagnosis of lung cancer in such individuals when considering such diagnostic methodologies.

There is therefore also clearly an unmet need for a diagnostic tools and refined FTIR analysis that is able to finely resolve and identify those patients with lung cancer at an early stage, especially amongst those individuals with an increased risk or likelihood of developing lung cancer due to the occurrence of other co-morbidities such as COPD.

Accordingly, we herein disclose a refined FTIR spectral waveform signature and analysis that can be used to diagnose and predict lung cancer and, significantly and superiorly, even amongst those individuals typically predicted to be at high risk of lung cancer. As disclosed herein, such high risk individuals can often otherwise exhibit changes to sputum that may lead to erroneous measure of sputum molecular changes, thereby skewing analysis and leading to inaccurate or inconclusive diagnosis when undertaking FTIR spectral analysis. Therefore, such signatures provide a robust and superior diagnostic analysis that pushes the diagnostic power of FTIR analysis in lung cancer to a level of increased sensitivity and specificity that is a pre-requisite in any clinical diagnostic test.

### STATEMENTS OF INVENTION

According to a first aspect of the invention there is provided a method for determining lung cancer in a human subject with a high risk or likelihood of developing lung cancer, the method comprising:
i) performing Fourier transform infrared (FTIR) spectral analysis of a sputum sample obtained from the subject to produce a sample spectrum;
ii) comparing the sample spectrum obtained in step i) with an FTIR spectrum from a control subject,
iii) wherein a difference between the spectrum produced from the sample and the control at one or more wavenumbers within one or more ranges selected from the group comprising: about 967 cm⁻¹, about 984 cm⁻¹, about 1024 cm⁻¹, about 1034 cm⁻¹, about 1055 cm 1, about 1079 cm⁻¹, about 1168 cm⁻¹, about 1388 cm⁻¹, about 1411 cm⁻¹, about 1440 cm⁻¹, about 1577 cm⁻¹, about 1656 cm⁻¹ is indicative of a subject suffering from lung cancer.

Remarkably, when analysing a sputum sample it has been found that these specific wavenumbers are able to accurately predict and diagnose lung cancer even in those individuals with high likelihood of developing lung cancer for example, but not limited to, COPD patients, smokers, persons previously exposed to asbestos, and persons who live in areas of high air pollution, and thus provide a robust and highly accurate method with heretofore undisclosed levels of sensitivity and specificity.

Reference herein lung cancer refers to any cancer or tumour originating from lung tissue including, but not limited to: non-small cell lung cell carcinomas such as adenocarcinoma, squamous-cell carcinoma, and large-cell carcinoma; small-cell lung carcinoma; adenosquamous carcinoma; mesothelioma; carcinoid tumours; bronchial gland carcinomas; sarcomatoid carcinomas.

As is known in the art, sputum refers to the coughed-up material (phlegm), typically secreted by goblet cells from the lower airways (trachea and bronchi). Sputum can be any colour including clear, white, yellow, green, pink or red and blood tinged which can result from different medical conditions. In addition to containing dead cells, foreign debris that is inhaled into the lung, and at times, bacteria, sputum contains white blood cells and other immune cells that protect the airway from infections. Contrary to FTIR analysis of the prior art, wherein a substantial degree of processing of the sputum is required e.g. preparation of cell pellets and heating of the sample, in a preferred method whole sputum, ideally dried, is utilised and subject to FTIR analysis. As will be appreciated by those skilled in the art, in this manner the sputum samples are advantageously tested without substantial sample pre-processing or preparation, other than sampling from the subject to be tested and optionally freezing (for storage purposes), thawing and/or drying (either by active process or merely by atmospheric drying). In addition to simplifying sample handling and processing times, it has been found that this preparation minimises presence of saliva in the sputum sample which can lead to spectral artefacts that may adversely affect diagnostic result.

Reference herein to a control sample refers to a sample that has been shown not to have lung cancer using any one or more conventional techniques for identifying same such as, but not limited to flexible bronchoscopy, CT-scan, X-ray, ultrasound, MRI or the like. As is disclosed herein, it has been found that, through rigorous analysis, a refined FTIR spectral waveform signature has been determined that can be used to diagnose and predict lung cancer and, significantly and superiorly, even amongst those individuals typically predicted to be at high risk of lung cancer. As will be appreciated by those skilled in the art, such high risk individuals can often otherwise exhibit changes to sputum that may lead to erroneous measure of sputum molecular changes that would skew analysis and so lead to inaccurate or inconclusive diagnosis when undertaking FTIR spectral analysis using wavenumber signatures of the prior art. Accordingly, and more preferably, said control sample is from a subject with a high risk or likelihood of developing lung cancer, such as but not limited to, persons with COPD, smokers, persons previously exposed to asbestos, and/or persons who live in areas of high air pollution, but shown not to have lung cancer as determined using any one or more conventional techniques for identifying same.

In a preferred method of the invention, FTIR analysis is undertaken in transmission mode. In contrast to reflectance modes (such as Attenuated Total Reflectance FTIR (ATR-FTIR)), wherein incident light is reflected and measured to provide spectral readings, in transmission mode the sample is placed directly into the infrared (IR) beam. As the IR beam passes through the sample, the transmitted energy is measured and a spectrum is generated. Whilst the quality of the data produced is comparable in different modes, readings can be affected at certain wavenumbers owing to the presence of water which if present can obscure the protein absorbance bands. In the present context, water vapour is easily trapped in sputum at the sputum/substrate interface as it dries onto the substrate. However, the affect from water vapour is decreased using transmission FTIR, due to the IR beam passing directly through the whole sample.

Reference herein to the term ‘about’ means plus or minus 5% and most preferably plus or minus 2%. For example, given the nature of the art it will be appreciated by those skilled in the field that there may be variation around recited wavenumbers owing to sample variability, for example, ±5 cm⁻¹, ±4 cm⁻¹, ±3 cm⁻¹, ±2 cm⁻¹, ±1 cm⁻¹.

As will be appreciated by those skilled in the art, in a preferred method of the invention the, or each, spectra preferably undergoes one or more conventional pre-processing steps prior to or following the comparison step to reduce the noise associated with the one or more spectra to provide the, or each, processed spectra. The pre-processing step(s) may comprise one or more of: background subtraction, and/or normalisation such as vector normalisation and/or baseline correction, or other method known to those skilled in the art. Preferably multiple output spectra are obtained and each spectrum is preferably subjected to one or more, preferably two or more, of wavenumber correction, baseline correction and vector normalisation.

In preferred embodiments, the, or each, processed spectra is then further processed to provide one or more dimensionally reduced spectrum such as, but not limited to, second derivative spectra with respect to wavenumber (or frequency). The or each dimensionally reduced spectrum/spectra is/are then compared to similarly dimensionally reduced control spectra.

In a preferred method, step iii) comprises or consists of observing a difference wherein an increase or decrease in absorbance at the same wavenumber or a shift in position of absorbance maxima or minima between wavenumbers between the spectrum produced from the sample and the control is indicative of a subject suffering from lung cancer. Most preferably, an increase or decrease in absorbance between wavenumbers between the spectrum produced from the sample and the control is observed. More preferably still, the method comprises or consists of observing a difference wherein an increased absorbance at a wavenumber selected from one or more of about 984 cm⁻¹, about 1034 cm⁻¹, 1055 cm⁻¹ and 1440 cm⁻¹ and/or wherein a decreased absorbance at a wavenumber selected from one or more of about 967 cm⁻¹, about 1024 cm⁻¹, about 1079 cm⁻¹, about 1168 cm⁻¹, about 1388 cm⁻¹, about 1411 cm⁻¹, about 1577 cm⁻¹, and 1656 cm⁻¹ in the sample when compared with the control is indicative of a subject suffering from lung cancer.

More preferably still, the said method comprises or consists of comparing the spectral absorbance at wavenumbers in any combination selected from the group comprising or consisting of: about 967 cm⁻¹, about 984 cm⁻¹, about 1024 cm⁻¹, about 1034 cm⁻¹, about 1055 cm⁻¹, about 1079 cm⁻¹, about 1168 cm⁻¹, about 1388 cm⁻¹, about 1411 cm⁻¹, about 1440 cm⁻¹, about 1577 cm⁻¹, about 1656 cm⁻¹, and more preferably a combination of at least two wavenumbers.

Yet more preferably still, said method comprises or consists of comparing the spectral absorbance at any one or more of the following combinations of wavenumbers: 984 cm⁻¹ and 967 cm⁻¹; 1024 cm⁻¹ and 967 cm⁻¹; 1055 cm⁻¹ and 967 cm⁻¹; 1079 cm⁻¹ and 967 cm⁻¹; 1411 cm⁻¹ and 967 cm⁻¹; 1577 cm⁻¹ and 967 cm⁻¹; 1656 cm⁻¹ and 967 cm⁻¹; 1079 cm⁻¹ and 1034 cm⁻¹; 1079 cm⁻¹ and 1034 cm⁻¹; 1079 cm⁻¹ and 1168 cm⁻¹; 1079 cm⁻¹ and 1388 cm⁻¹; and/or 1079 cm⁻¹ and 1440 cm⁻¹. In exemplary embodiments, spectral absorbance at 1079 cm⁻¹ and 1168 cm⁻¹ or at 1079 cm⁻¹ and 967 cm⁻¹ is compared.

In preferred embodiments, second derivative absorbances with respect to wavenumber are compared according to one or more of the following equations, wherein x represents the second derivative absorbance at a first wavenumber, y represents the second derivative absorbance at a second wavenumber and wherein cancer is indicated if said equation is satisfied.

| Wavenumber (cm⁻¹) | | |
|---|---|---|
| x | y | Equation |
| 984 | 967 | y > (−4.65*x) − 0.00018 |
| 1024 | 967 | y > (−0.83*x) − 0.00005 |
| 1055 | 967 | y ≤ (0.80*x) + 0.00004 |
| 1079 | 967 | y > (−0.56*x) − 0.00013 |
| 1411 | 967 | y > (−0.66*x) − 0.00004 |
| 1577 | 967 | y < (1.07*x) + 0.00008 |
| 1656 | 967 | y > (−17.78*x) − 0.00081 |
| 1079 | 1034 | y > (−0.43*x) − 0.00010 |
| 1079 | 1168 | y > (−0.73*x) − 0.00013 |
| 1079 | 1388 | y > (−0.47*x)−0.00011 |
| 1079 | 1440 | y > (−0.70*x) − 0.00011 |

More preferably, second derivative absorbances with respect to wavenumber are compared according to one or more of the following equations, wherein x represents the second derivative absorbance at a first wavenumber, y represents the second derivative absorbance at a second wavenumber and wherein cancer is indicated if said equation is satisfied.

| Wavenumber (cm⁻¹) | | |
|---|---|---|
| x | y | Equation |
| 984 | 967 | y > (−4.651163*x) − 0.000179 |
| 1024 | 967 | y > (−0.83333*x) − 0.000047 |
| 1055 | 967 | y ≤ (0.80357*x) + 0.000039 |
| 1079 | 967 | y > (−0.55556*x) − 0.00013 |
| 1411 | 967 | y > (−0.6579*x) − 0.00004 |
| 1577 | 967 | y < (1.07143*x) + 0.000079 |
| 1656 | 967 | y > (−17.77889*x) − 0.000811 |
| 1079 | 1034 | y > (−0.4286*x) − 0.0001 |
| 1079 | 1168 | y > (−0.72619*x) − 0.000125 |
| 1079 | 1388 | y > (−0.4699999998*x) − |
| | | 0.00010600000004 |
| 1079 | 1440 | y > (−0.7001*x) − 0.000111 |

Alternatively, as will be readily appreciated by those skilled in the art, the comparison of the second derivative absorbance at a first wavenumber with the second derivative absorbance at a second wavenumber can be considered relative to one another in the alternative arrangement using a mathematically rearranged derivative of one or more of the equations identified above, and used to indicate cancer to equal effect.

According to a further aspect of the invention, there is provided a method for determining lung cancer in a human subject with a high risk or likelihood of developing lung cancer, the method comprising:
i) performing Fourier transform infrared (FTIR) spectral analysis of a sputum sample obtained from the subject to produce a sample spectrum;
ii) processing the spectrum obtained in step i) to provide one or more second derivative reduced spectra with respect to wavenumber (or frequency);
iii) measuring the spectral absorbance at any one or more of the following combinations of wavenumbers: 984 cm⁻¹ and 967 cm⁻¹; 1024 cm⁻¹ and 967 cm⁻¹; 1055 cm⁻¹ and 967 cm⁻¹; 1079 cm⁻¹ and 967 cm⁻¹; 1411 cm⁻¹ and 967 cm⁻¹; 1577 cm⁻¹ and 967 cm⁻¹; 1656 cm⁻¹ and 967 cm⁻¹; 1079 cm⁻¹ and 1034 cm⁻¹; 1079 cm⁻¹ and 1034 cm⁻¹; 1079 cm⁻¹ and 1168 cm⁻¹; 1079 cm⁻¹ and 1388 cm⁻¹; and/or 1079 cm⁻¹ and 1440 cm⁻¹; and
iv) comparing the spectral absorbance(s) with respect to wavenumber according to one or more of the following equations, wherein x represents the second derivative absorbance at a first wavenumber, y represents the second derivative absorbance at a second wavenumber and wherein cancer is indicated if said equation is satisfied.

| Wavenumber (cm⁻¹) | | |
|---|---|---|
| x | y | Equation |
| 984 | 967 | y > (−4.65*x) − 0.00018 |
| 1024 | 967 | y > (−0.83*x) − 0.00005 |
| 1055 | 967 | y ≤ (0.80*x) + 0.00004 |
| 1079 | 967 | y > (−0.56*x) − 0.00013 |
| 1411 | 967 | y > (−0.66*x) − 0.00004 |
| 1577 | 967 | y < (1.07*x) + 0.00008 |
| 1656 | 967 | y > (−17.78*x) − 0.00081 |
| 1079 | 1034 | y > (−0.43*x) − 0.00010 |
| 1079 | 1168 | y > (−0.73*x) − 0.00013 |
| 1079 | 1388 | y > (−0.47*x) −0.00011 |
| 1079 | 1440 | y > (−0.70*x) − 0.00011 |

More preferably still, step iv) of said method comprises comparing absorbances with respect to wavenumber are according to one or more of the following equations, wherein x represents the second derivative absorbance at a first wavenumber, y represents the second derivative absorbance at a second wavenumber and wherein cancer is indicated if said equation is satisfied.

| Wavenumber (cm⁻¹) | | |
|---|---|---|
| x | y | Equation |
| 984 | 967 | y > (−4.651163*x) − 0.000179 |
| 1024 | 967 | y > (−0.83333*x) − 0.000047 |
| 1055 | 967 | y ≤ (0.80357*x) + 0.000039 |
| 1079 | 967 | y > (−0.55556*x) − 0.00013 |
| 1411 | 967 | y > (−0.6579*x) − 0.00004 |
| 1577 | 967 | y < (1.07143*x) + 0.000079 |
| 1656 | 967 | y > (−17.77889*x) − 0.000811 |
| 1079 | 1034 | y > (−0.4286*x) − 0.0001 |
| 1079 | 1168 | y > (−0.72619*x) − 0.000125 |
| 1079 | 1388 | y > (−0.4699999998*x) − |
| | | 0.00010600000004 |
| 1079 | 1440 | y > (−0.7001*x) − 0.000111 |

Alternatively, as will be readily appreciated by those skilled in the art, the comparison of the second derivative absorbance at a first wavenumber with the second derivative absorbance at a second wavenumber can be considered relative to one another in the alternative arrangement using a mathematically rearranged derivative of one or more of the equations identified above, and used to indicate cancer to equal effect.

According to a further aspect of the invention, there is provided a method for monitoring the progression of lung cancer in a human subject comprising repeating one or more of the afore, and ideally the same, method(s) periodically.

Ideally, FTIR analysis is correlated with known lung cancer staging techniques such that of a simple in vitro assay or biopsy used to reliably inform a clinician about, not only the existence of a lung cancer, but also its stage or progression.

As will be appreciated by those skilled in the art, in the above method of the invention comparing spectral absorbance with respect to wavenumber from the sample and/or analyzing relative spectral difference(s) between the spectrum produced from the sample and the control sample at one or more wavenumbers within one or more ranges disclosed herein can be used to assess how effective a treatment regimen is working, for example, by assaying the absorbance levels during the course of a given therapy to determine if there is a change in the absorbance in response to said treatment.

Additionally, or alternatively, there is provided a method for treating lung cancer comprising performing any one of the afore methods and then, depending upon the outcome of the method, undertaking a suitable or selected course of treatment.

According to a further aspect of the invention there is provided a kit for use in determining lung cancer in a sputum sample from human subject with a high risk or likelihood of developing lung cancer, said kit comprising:
a) an FTIR machine for performing spectral analysis of a sputum sample obtained from the subject;
b) a processing unit, wherein said processing unit processes the spectrum from the sample according to a method as defined herein; and
c) an output unit arranged to provide an output indicative of a subject suffering from lung cancer in the subject according to a determination made by the processing unit in step b).

In a preferred kit of the invention, FTIR analysis is undertaken in transmission mode.

In yet a further preferred kit of the invention, the kit further comprises a collection means for collecting the sputum samples, ideally provided as an infrared transparent slide.

Yet more preferably still, said kit further comprises a sample or slide holder to keep the slide in place during measurement of absorbance.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

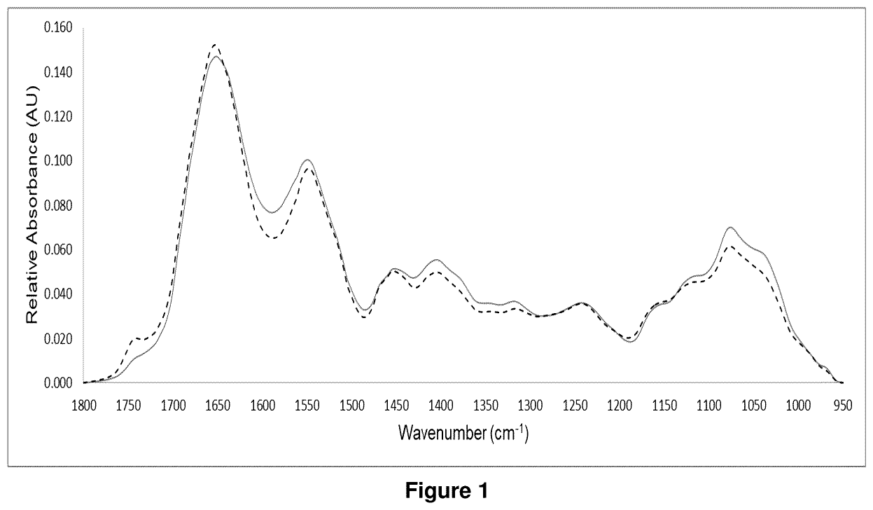

FIG. 1. Vector-normalised, baseline-corrected absorbance spectra from 1800-950 cm⁻¹ of cancer (black dashed), COPD (grey solid) cohorts. Peaks and troughs were differences between cancer and non-cancer are visible are easily identifiable at approximately 1740, 1650, 1590, 1410 and 1075 cm⁻¹;

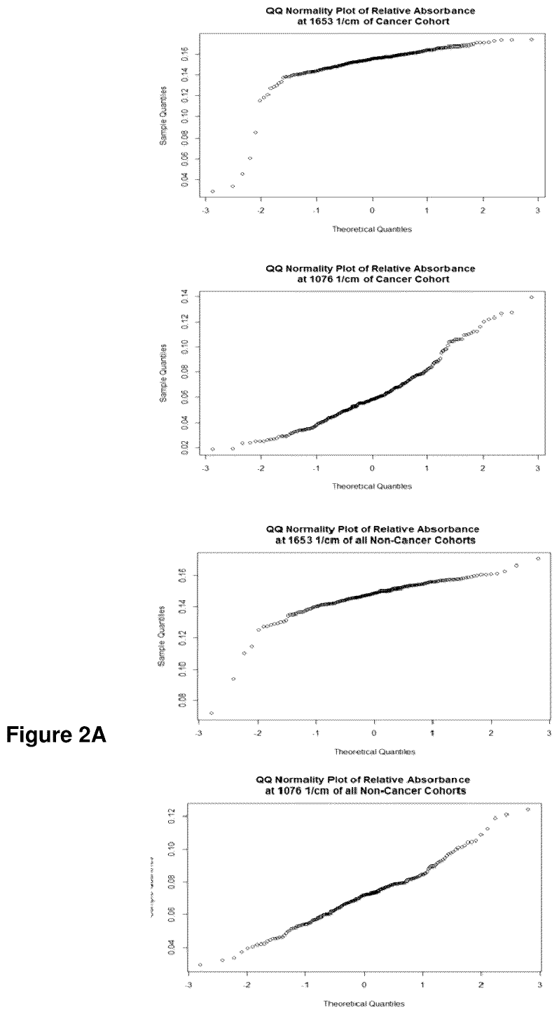

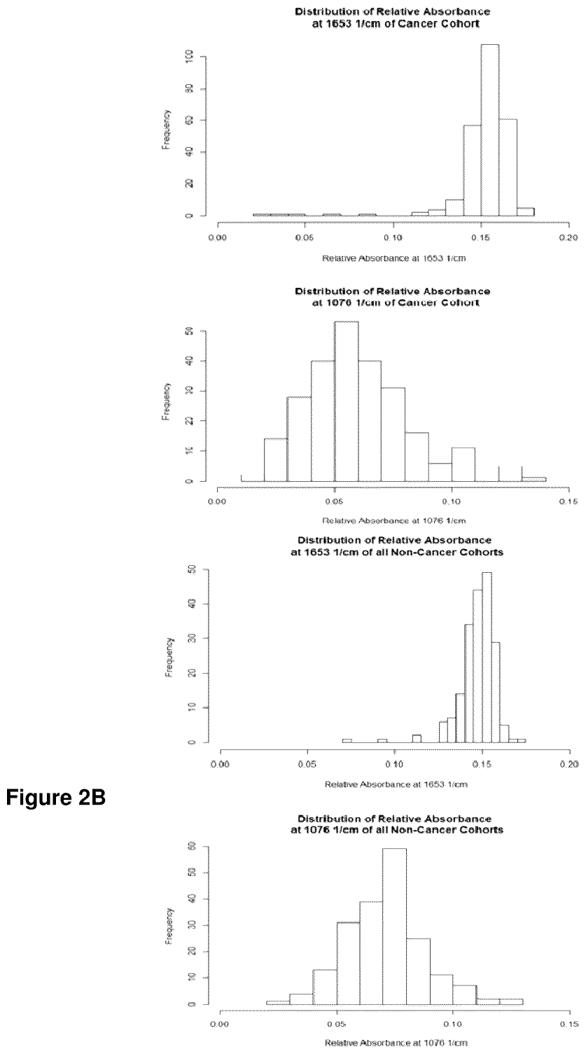

FIGS. 2A-2B. FIG. 2A) QQ Normality plots and FIG. 2B) frequency histograms of distribution of absorbencies at 1653 cm⁻¹ and 1076 cm⁻¹. Plots of distribution at 1653 cm⁻¹ show a heavy-tailed distribution. Distribution at 1076 cm⁻¹ is closer to normality but the cancer cohort shows a light-tailed distribution and the control cohorts show a light skewing;

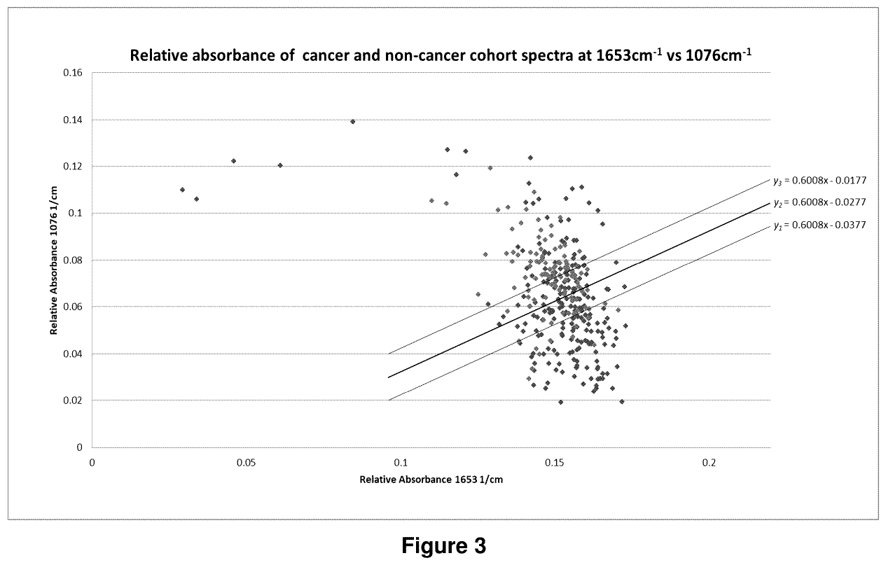

FIG. 3. Vector-normalised baseline-corrected absorbencies at 1076 cm⁻¹ and 1653 cm⁻¹ of all cancer (red), and COPD (green)

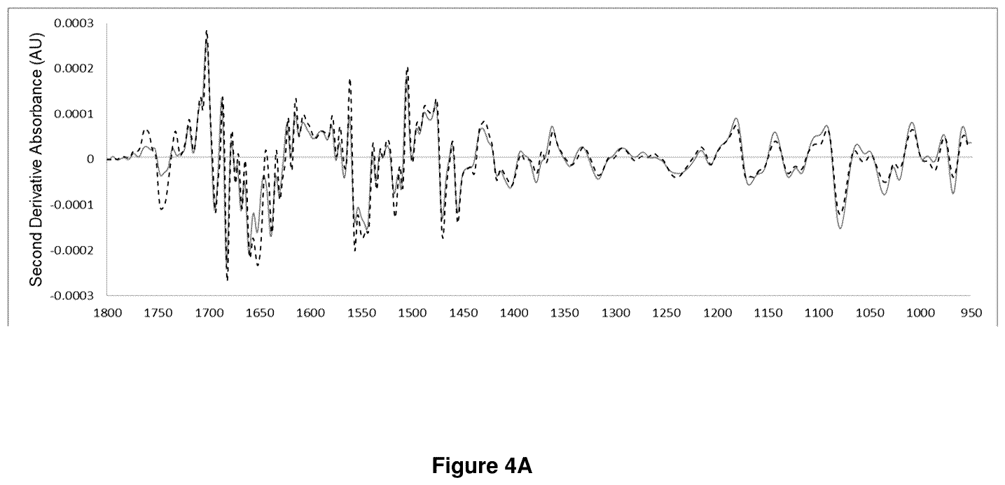

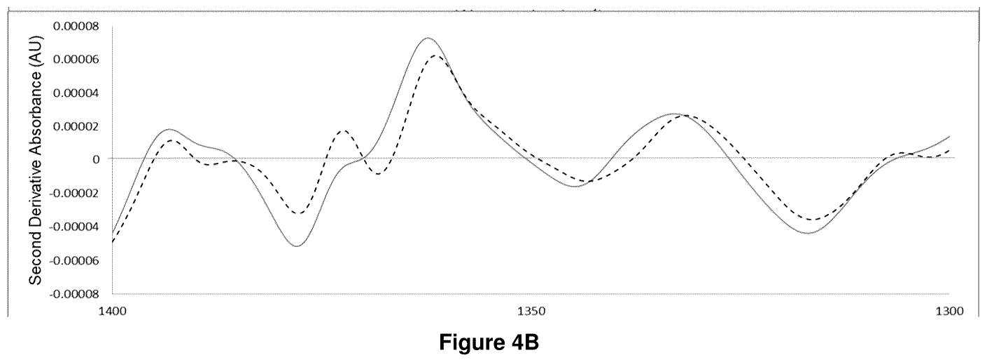

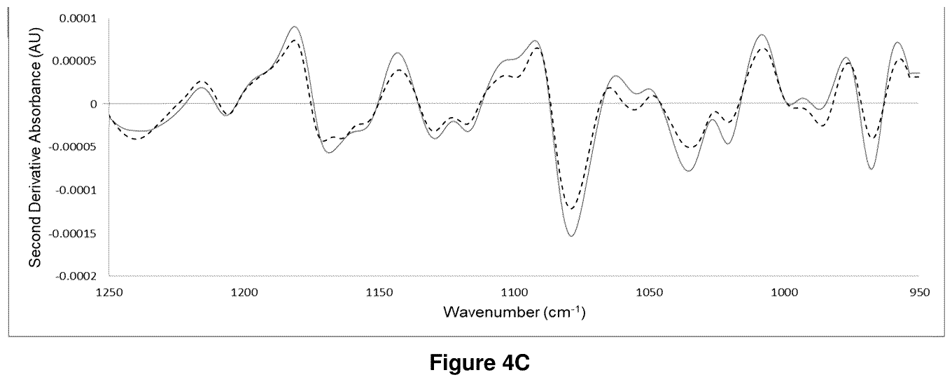

FIGS. 4A-4C. Second derivative spectra from (FIG. 4A) 1800-950 cm⁻¹ (FIG. 4B) 1400-1300 cm⁻¹, and (FIG. 4C) 1250-950 cm⁻¹ of cancer, COPD cohorts (colours as in FIG. 1).

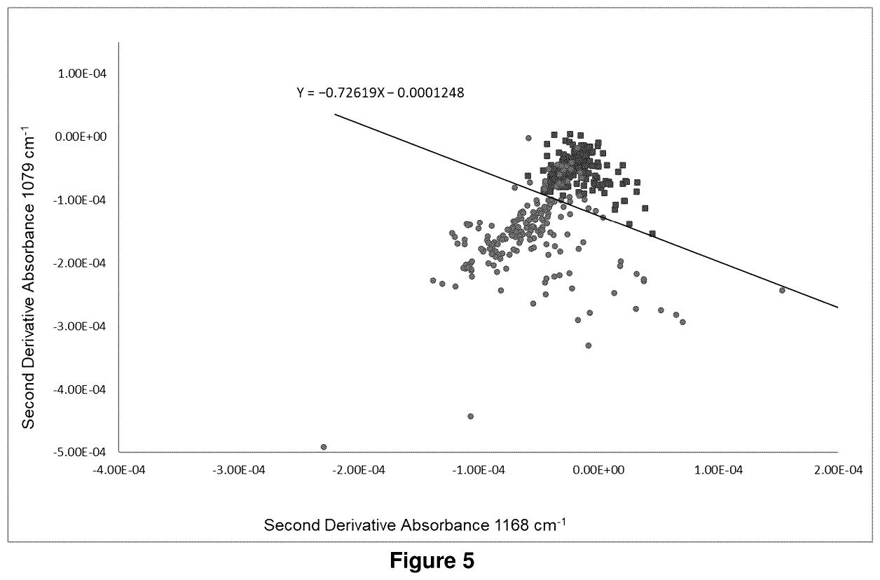

FIG. 5. Comparison of second derivative intensities at 1168 cm⁻¹ and 1079 cm⁻¹ in cancer (red) and COPD (green) patient spectra. The linear regressor separates the patient clusters and the associated equation is used as the predictor.

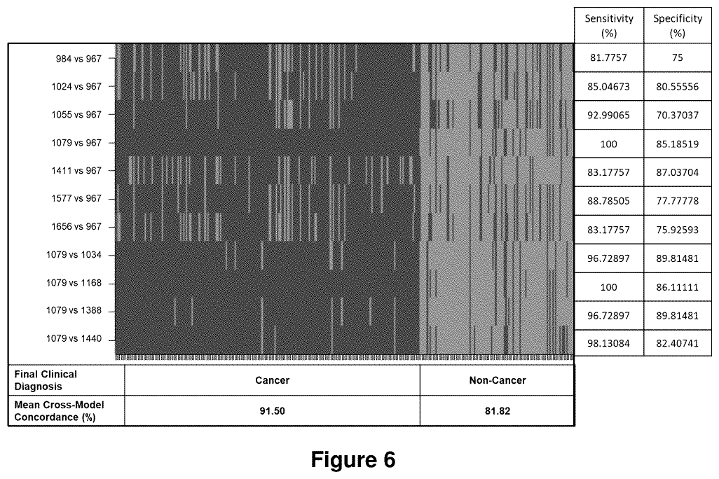

FIG. 6. Two-dimensional heatmap of predictions by each regression model, with sensitivities and specificities for each model shown. A prediction of cancer is shown as red, and a prediction of COPD is shown as green.

Table 1. Results from Shapiro-Wilk test for normality of distribution of absorbencies at 1740, 1653, 1589, 1410 and 1076 cm⁻¹ in cancer and non-cancer control cohorts. P<0.05 suggests that the null hypothesis of normally distributed data can be rejected and the data are non-normally distributed. *P>0.05, the null hypothesis cannot be rejected, the data are normally distributed;

Table 2. Results of significance testing, comparing the normalized absorbencies at each wavenumber between the cancer and non-cancer cohorts with a Mann-Whitney U test; all wavenumbers tested were shown to be highly-significantly different between the patient groups;

Table 3. Sensitivity and specificity scores for determining lung cancer from non-cancer control groups based on the equations of the three lines shown in FIG. 3;

Table 4. Average major peak positions within the glycogen-rich region from a subset of 60 randomly selected lung cancer patient second-derivative spectra. Standard deviation and variance for each peak has been calculated and the lowest values are highlighted;

Table 5. Average major peak positions within the glycogen-rich region from all COPD patient second-derivative spectra. Standard deviation and variance for each peak has been calculated and the lowest values are highlighted;

Table 6. Summary of two-dimensional linear model performances, with equations, and model performance characteristics shown;

Table 7. Mean second derivative absorbances for cancer and COPD groups.

### Methods

### Patient Recruitment

All patients provided informed consent for their samples to be used in future research.

The Medlung observational study (REC No: 05/MWM01/75) recruited patients who attended bronchoscopy clinics across the UK under suspicion of lung cancer and were subsequently given a final clinical diagnosis of either lung cancer or non-cancer. Patients were referred to the clinic and gave informed consent before providing a sample of spontaneous sputum. The patients' final clinical diagnosis and histology was recorded. Confirmed cancer cases and confirmed COPD cases make up the “Cancer”, “COPD control” cohorts respectively.

In total, raw sputum samples collected from 214 lung cancer patients with fully confirmed histologies, and 108 COPD patients as a higher-risk control group were used in this study.

Each sputum sample was stored at −80° C. until time of spectrum generation.

### Spectrum Acquisition and Sample Processing

Transmission-FTIR (t-FTIR) was performed on raw sputum samples using a Bruker Vertex 70 with high throughput attachment (HTS-XT), KBr beamsplitter, and DGTS detector. Prior to all measurements, ninety-six well silicon plates (Bruker) were cleaned in 70% ethanol and rinsed with dH2O three times and air dried. The sputum samples were allowed to thaw-out in their sealed containers and reach room temperature for at least one-hour prior to analysis. Raw (i.e. without further labelling or sample purification), thawed-out sputum samples were pipetted (2 μl) directly onto the plates in triplicate and allowed to dry under atmospheric conditions. Once dry, spectra were generated at 32 scans per spectrum, with a fresh background spectrum taken between each sample spectrum. Each 96-well plate was scanned in triplicate, to give a total of 9 replicate spectra per sample.

### Spectrum Processing

All spectra underwent quality analysis prior to processing. All sample replicates were averaged before vector-normalisation and baseline-correction using the OPUS 7.5 (Bruker) in-built baseline-correction, vector-normalisation algorithms. Second derivative spectra were generated using the Savitzky-Golay method with 9 smoothing points. This approach allowed us to resolve broad, overlapping bands into individual bands thus increasing the accuracy of analysis. Peak peaking analysis was carried out using the in-built peak peaking algorithm in OPUS, set to a 5% threshold.

### Statistical Analysis

Statistical tests were carried out using the programming environment R. Statistical significance was calculated using the non-parametric Mann-Whitney U Test, at a level of 0.05. Multiple hypothesis testing was carried out using Bonferroni correction. Normality of data was established using a Shapiro-Wilk test and visualisations through QQ-plots and histograms.

### Model Building

Two dimensional linear models were produced comparing second derivative absorbencies at specific wavenumbers. Model performance was quantified using sensitivity and specificity.

### Results

### 1. FTIR Spectrum of Cancer Sputum

Shown in FIG. 1 are vector-normalised, baseline-corrected average absorbance spectra of cancer (black dashed) and COPD (solid grey) cohorts. Whilst the average spectra appear to be highly similar, with little variation in peak position or relative absorbance, subtle differences can be identified.

For example, the relative intensity of multiple peaks and troughs can be seen to be different between cancer and non-cancer average spectra. For example, the relative absorbance at amide I (˜1653 cm⁻¹) is higher in the cancer average spectrum, and the major glycogen peak (˜1076 cm⁻¹) is lower compared to the non-cancer average spectra. The proposed vibrational mode of 1653 cm⁻¹ is C═O stretching from a protein source. The proposed vibrational mode of the glycogen peak at ˜1076 cm⁻¹ is C—O stretching, from the alcohol groups found within individual monosaccharide moieties throughout the sputum. This suggests that overall glycosylation compared to protein content in lung cancer sputum could be reduced, compared to non-cancer sputum. Additionally, the trough between amide I and amide II at around 1589 cm⁻¹ of the cancer spectrum appears to be lower than all of non-cancer spectra, whilst the amide I peak shows a greater relative intensity than the non-cancer amide I peaks. This may suggest a reduction in the levels of amino-sugars such as sialic acid, N-acetylgalactosamine (GalNAc) or N-acetylglucosamine (GlcNAc) relative to the levels of protein present in lung cancer sputum.

A series of wavenumbers (1740, 1653, 1589, 1410 and 1076 cm⁻¹) were identified as potential markers that could be used to discriminate between cancer and non-cancer sputum, due to clear visible differences in the average spectra (FIG. 1). Normality and significance testing was subsequently performed.

### 2. Normality and Significance Testing

Normality testing was carried out to ascertain how the absorbencies at each wavenumber were distributed across the cancer and control cohorts. A Shapiro-Wilk (SW) test for normality on five wavenumbers which correspond with positions of major peaks and troughs was initially carried out, and the results are summarised in Table 1. The results suggested that the null hypothesis that the data were drawn from a normally distributed population can be rejected, therefore indicating the data are non-normally distributed.

QQ-plots and histograms were drawn to visualise distribution (FIGS. 2A-2B). The distribution of absorbencies at 1653 cm⁻¹ was shown to be heavily tailed, and the distribution at 1076 cm⁻¹ can be said to be closer to normality, but still demonstrated a light skewing in both cancer and control cohorts. Therefore, combining these results with the Shapiro-Wilk normality results, the data can be said to be non-normally distributed. Thus, non-parametric statistical testing was carried out to determine significance of differences between absorbencies at wavenumbers of interest.

As all wavenumbers tested were shown to be drawn from non-normally distributed data. The non-parametric Mann-Whitney U test was carried out to assess the statistical significance of any differences between the mean absorbencies (Table 2).

FIG. 3 demonstrates how the different patient cohorts cluster and separate from each other based on the absorbance at 1076 and 1653 cm⁻¹, as well as showing the overall trend within each patient cohort. There appears to be a small trend for cancer sputum spectra (red) to show a slight increase in relative absorption at 1653 cm⁻¹ alongside a slight decrease in relative absorption at 1076 cm⁻¹, compared to the non-cancer sputum spectra (green).

Despite these trends, the discriminatory power of these wavenumbers is poor. The calculated sensitivity and specificity scores for a model based on normalized absorbencies at 1653 cm⁻¹ and 1076 cm⁻¹ are shown in Table 3. The cancer and non-cancer patient cohorts exhibit a large overlap, so the intercept of the linear separator was modified to optimise sensitivity and specificity scores. The most accurate equation was determined to be y₂, with sensitivity and specificity of 61.11% and 71.65% respectively. The other models demonstrated stronger specificity but poor sensitivity (y₁) or vice versa (y₃).

### 3. Second Derivative Absorbance Models

Second derivative spectra were calculated from the vector-normalised, baseline-corrected average spectra (FIGS. 4A-4C). A second-derivative spectrum is used to increase the sensitivity for peak finding by calculating the rate-of-change across a small window of the spectrum. In this way, the resolution of small peaks and shoulders within larger peaks is increased.

The average second-derivative spectra were closely examined to identify regions of the spectra that could be used to distinguish cancer from non-cancer. Wavenumbers were identified having good discriminatory potential for lung cancer sputum for analysis.

Two dimensional linear models were generated to examine how the second-derivative absorbencies of interest at specific wavenumbers could separate the cohorts within two dimensions. The second-derivative absorbance at 967 cm⁻¹ was initially chosen as a standard for plotting against other wavenumbers. This was because it was readily identifiable in all spectra and was calculated to have the lowest standard deviation and variance compared to all other major peaks detected in the cancer (Table 4), and COPD (Table 5) data sets.

A series of two dimensional linear models were built and tested for sensitivity and specificity for determining lung cancer from COPD. These are summarised in Table 6 and the best performing model (1168 vs 1079 cm⁻¹) is shown in FIG. 5. The predictions for each model are shown in the heatmap in FIG. 6.

TABLE 1

| Wavenumber (cm⁻¹⁻) | Cancer | Non−Cancer |
|---|---|---|
| 1740 | <2.2e−16 | 6.68E−15 |
| 1653 | <2.2e−16 | 2.06E−14 |
| 1589 | 0.00102 | 0.006305 |
| 1410 | 3.11E−08 | 8.18E−06 |
| 1076 | 6.38E−07 | 0.0723 |

TABLE 2

| | Mean Cancer | Mean Non-Cancer | |
|---|---|---|---|
| Wavenumber | Spectrum | Spectrum | |
| (cm⁻¹) | Absorbance | Absorbance | p-Value |
| 1740 | 0.020078 | 0.011667 | 7.185e−15 |
| 1653 | 0.152049 | 0.146985 | 1.02*10 − 12 |
| 1589 | 0.065135 | 0.078781 | <2.2e−16 |
| 1411 | 0.048932 | 0.055263 | 1.958e−09 |
| 1076 | 0.061379 | 0.071092 | 2.45*10 − 9 |

TABLE 3
1076 vs 1653 cm⁻¹

| y₁ = 0.6008x − 0.0377 | | y₂ = 0.6008x − 0.0277s | | y₃ = 0.6008x − 0.0177 | |
|---|---|---|---|---|---|
| True Positives | 109 | True Positives | 154 | True Positives | 188 |
| False Positives | 25 | False Positives | 55 | False Positives | 104 |
| True Negatives | 169 | True Negatives | 139 | True Negatives | 90 |
| False Negatives | 143 | False Negatives | 98 | False Negatives | 64 |
| Sensitivity (%) | 43.26 | Sensitivity | 61.11 | Sensitivity | 74.60 |
| Specificity (%) | 87.11 | Specificity | 71.65 | Specificity | 46.39 |

TABLE 4

| | Mean Peak Position | | | | | | | | |
|---|---|---|---|---|---|---|---|---|---|
| | 1171 | 1130 | 1079 | 1057 | 1035 | 1020 | 998 | 986 | 968 |
| Mode | 1171 | 1130 | 1079 | 1057 | 1036 | 1021 | 999 | 984 | 968 |
| Std Dev | 1.686 | 1.298 | 1.341 | 1.68 | 2.58 | 2.050 | 2.39 | 3.19 | 1.13 |
| Min peak | 1168 | 1127 | 1071 | 1054 | 1028 | 1014 | 993 | 976 | 964 |
| position | | | | | | | | | |
| Max peak | 1174 | 1132 | 1081 | 1060 | 1040 | 1022 | 1002 | 991 | 970 |
| position | | | | | | | | | |
| Variance | 2.783 | 1.631 | 1.767 | 2.73 | 6.51 | 4.07 | 5.55 | 9.86 | 1.25 |

TABLE 5

| | Mean Peak Position | | | | | | | | |
|---|---|---|---|---|---|---|---|---|---|
| | 1168 | 1130 | 1079 | 1054 | 1036 | 1021 | 998 | 985 | 968 |
| Mode | 1168 | 1130 | 1079 | 1055 | 1035 | 1021 | 999 | 984 | 968 |
| Std Dev | 2.15 | 1.46 | 1.91 | 2.99 | 1.53 | 0.64 | 0.69 | 1.34 | 0.41 |
| Min peak | 1161 | 1125 | 1074 | 1050 | 1031 | 1020 | 997 | 984 | 967 |
| position | | | | | | | | | |
| Max peak | 1172 | 1132 | 1085 | 1060 | 1039 | 1022 | 1000 | 990 | 969 |
| position | | | | | | | | | |
| Variance | 4.52 | 2.07 | 3.54 | 8.18 | 2.27 | 0.40 | 0.46 | 1.73 | 0.16 |

TABLE 6

| | | | | True | True | False | False | Sensitivity | Specificity | Accuracy |
|---|---|---|---|---|---|---|---|---|---|---|
| x | | y | Equation | Positives | Negatives | Positives | Negatives | (%) | (%) | (%) |
| 984 | Vs | 967 | y > (−4.651163*x) − | 175 | 81 | 27 | 39 | 81.78% | 75.00% | 79.50% |
| | | | 0.000179 | | | | | | | |
| 1024 | Vs | 967 | y > (−0.83333*x) − | 182 | 87 | 21 | 32 | 85.05% | 80.56% | 83.54% |
| | | | 0.000047 | | | | | | | |
| 1055 | Vs | 967 | y ≤ (0.80357*x) + | 180 | 83 | 25 | 34 | 84.11% | 76.85% | 81.68% |
| | | | 0.000039 | | | | | | | |
| 1079 | Vs | 967 | y > (−0.55556*x) − | 214 | 92 | 16 | 0 | 100.00% | 85.19% | 95.03% |
| | | | 0.00013 | | | | | | | |
| 1411 | Vs | 967 | y > (−0.6579*x) − | 178 | 94 | 14 | 36 | 83.18% | 87.04% | 84.47% |
| | | | 0.00004 | | | | | | | |
| 1577 | Vs | 967 | y < (1.07143*x) + | 190 | 84 | 24 | 24 | 88.79% | 77.78% | 85.09% |
| | | | 0.000079 | | | | | | | |
| 1656 | Vs | 967 | y > (−17.77889*x) − | 178 | 82 | 26 | 36 | 83.18% | 75.93% | 80.75% |
| | | | 0.000811 | | | | | | | |
| 1079 | Vs | 1034 | y > (−0.4286*x) − | 198 | 98 | 10 | 16 | 92.52% | 90.74% | 91.93% |
| | | | 0.0001 | | | | | | | |
| 1079 | Vs | 1168 | y > (−0.72619*x) − | 214 | 93 | 15 | 0 | 100.00% | 86.11% | 95.34% |
| | | | 0.0001248 | | | | | | | |
| 1079 | Vs | 1388 | y > (−0.4699999998*x) − | 185 | 102 | 6 | 29 | 86.45% | 94.44% | 89.13% |
| | | | 0.00010600000004 | | | | | | | |
| 1079 | Vs | 1440 | y > (−0.7001*x) − | 210 | 89 | 19 | 4 | 98.13% | 82.41% | 92.86% |
| | | | 0.000111 | | | | | | | |

TABLE 7

| | Mean | | | Mean | | |
|---|---|---|---|---|---|---|
| Wavenumber | Cancer | Min | Max | COPD | Min | Max |
| 967 | −1.6E−05 | −8.5E−05 | 0.000107 | −6.5E−05 | −0.00011 | 0.000144 |
| 984 | −1.1E−05 | −0.00028 | 2.23E−05 | 4.54E−06 | −0.00017 | −3.6E−06 |
| 1024 | −4.6E−06 | −3.9E−05 | 6.95E−05 | −3.3E−05 | −0.00017 | 8.86E−05 |
| 1034 | −1.9E−05 | −9.7E−05 | 3.77E−05 | −7.9E−05 | −0.00017 | 0.000109 |
| 1055 | −2.6E−06 | −9.9E−05 | 5.27E−05 | 2.08E−05 | −0.00033 | −4.6E−05 |
| 1079 | −5E−05 | −0.00015 | 3.95E−06 | −0.00016 | −0.00014 | 0.00007 |
| 1168 | −1.7E−05 | −5.9E−05 | 0.000045 | −5.1E−05 | −9.4E−06 | 7.06E−05 |
| 1388 | −2.2E−06 | −0.00014 | 0.000062 | 7.64E−06 | −5.1E−05 | 4.79E−05 |
| 1411 | −1.2E−05 | −0.00019 | 1.78E−05 | −3.3E−05 | −0.00013 | 9.99E−06 |
| 1440 | −1.5E−05 | −7E−05 | 0.000049 | −8.2E−06 | −7.3E−05 | 0.000102 |
| 1577 | 2.71E−05 | −4.8E−05 | 0.000112 | 3.99E−05 | −2.1E−05 | 8.26E−05 |
| 1656 | −8.5E−05 | −0.00041 | 0.000141 | −0.00014 | −0.00118 | 0.000229 |

1. A method for determining lung cancer in a human subject with a high risk or likelihood of developing lung cancer, the method comprising:
i) performing Fourier transform infrared (FTIR) spectral analysis of a sputum sample obtained from the subject to produce a sample spectrum;
ii) processing the spectrum obtained in step i) to provide one or more second derivative spectra with respect to wavenumber (or frequency);
iii) measuring the spectral absorbance at any one or more of the following combinations of wavenumbers: 984 cm-1 and 967 cm-1; 1024 cm-1 and 967 cm-1; 1055 cm-1 and 967 cm-1; 1079 cm-1 and 967 cm-1; 1411 cm-1 and 967 cm-1; 1577 cm-1 and 967 cm-1; 1656 cm-1 and 967 cm-1; 1079 cm-1 and 1034 cm-1; 1079 cm-1 and 1034 cm-1; 1079 cm-1 and 1168 cm-1; 1079 cm-1 and 1388 cm-1; and/or 1079 cm-1 and 1440 cm-1; and
iv) comparing the spectral absorbance(s) with respect to wavenumber according to one or more of the following equations or a mathematically rearranged derivative thereof, wherein x represents the second derivative absorbance at a first wavenumber, y represents the second derivative absorbance at a second wavenumber and wherein cancer is indicated if said equation is satisfied:

| Wavenumber (cm⁻¹) | | |
|---|---|---|
| x | y | Equation |
| 984 | 967 | y > (−4.65*x) − 0.00018 |
| 1024 | 967 | y > (−0.83*x) − 0.00005 |
| 1055 | 967 | y ≤ (0.80*x) + 0.00004 |
| 1079 | 967 | y > (−0.56*x) − 0.00013 |
| 1411 | 967 | y > (−0.66*x) − 0.00004 |
| 1577 | 967 | y < (1.07*x) + 0.00008 |
| 1656 | 967 | y > (−17.78*x) − 0.00081 |
| 1079 | 1034 | y > (−0.43*x) − 0.00010 |
| 1079 | 1168 | y > (−0.73*x) − 0.00013 |
| 1079 | 1388 | y > (−0.47*x) − 0.00011 |
| 1079 | 1440 | y > (−0.70*x) − 0.00011 |

2. A method for determining lung cancer in a human subject with a high risk or likelihood of developing lung cancer, the method comprising:
i) performing Fourier transform infrared (FTIR) spectral analysis of a sputum sample obtained from the subject to produce a sample spectrum;
ii) comparing the sample spectrum obtained in step i) with an FTIR spectrum from a control subject,
iii) wherein a difference between the spectrum produced from the sample and the control at one or more of the following combinations of wavenumbers: 984 cm-1 and 967 cm-1; 1024 cm-1 and 967 cm-1; 1055 cm-1 and 967 cm-1; 1079 cm-1 and 967 cm-1; 1411 cm-1 and 967 cm-1; 1577 cm-1 and 967 cm-1; 1656 cm-1 and 967 cm-1; 1079 cm-1 and 1034 cm-1; 1079 cm-1 and 1034 cm-1; 1079 cm-1 and 1168 cm-1; 1079 cm-1 and 1388 cm-1; and/or 1079 cm-1 and 1440 cm-1 is indicative of a subject suffering from lung cancer.

3. The method according to claim 2 wherein second derivative absorbances with respect to wavenumber are compared according to one or more of the following equations or a mathematically rearranged derivative thereof, wherein x represents the second derivative absorbance at a first wavenumber, y represents the second derivative absorbance at a second wavenumber and wherein cancer is indicated if said equation is satisfied:

| Wavenumber (cm⁻¹) | | |
|---|---|---|
| x | y | Equation |
| 984 | 967 | y > (−4.65*x) − 0.00018 |
| 1024 | 967 | y > (−0.83*x) − 0.00005 |
| 1055 | 967 | y ≤ (0.80*x) + 0.00004 |
| 1079 | 967 | y > (−0.56*x) − 0.00013 |
| 1411 | 967 | y > (−0.66*x) − 0.00004 |
| 1577 | 967 | y < (1.07*x) + 0.00008 |
| 1656 | 967 | y > (−17.78*x) − 0.00081 |
| 1079 | 1034 | y > (−0.43*x) − 0.00010 |
| 1079 | 1168 | y > (−0.73*x) − 0.00013 |
| 1079 | 1388 | y > (−0.47*x) − 0.00011 |
| 1079 | 1440 | y > (−0.70*x) − 0.00011 |

4. The method according to claim 2, wherein step iii) comprises or consists of observing a difference wherein an increase or decrease in absorbance at the same wavenumber or a shift in position of absorbance maxima or minima between wavenumbers between the spectrum produced from the sample and the control is indicative of a subject suffering from lung cancer.

5. The method according to claim 1, wherein said sputum sample is whole sputum.

6. The method according to claim 2, wherein said control sample is from a subject with a high risk or likelihood of developing lung cancer.

7. The method according to claim 1, wherein FTIR spectral analysis is undertaken in transmission mode.

8. The method according to claim 1, wherein the, or each, spectra undergoes one or more conventional pre-processing step(s) prior to or following the comparison step to reduce the noise associated with the one or more spectra to provide the, or each, processed spectra.

9. The method according to claim 8 wherein the one or more pre-processing step(s) are selected from the group comprising: background subtraction and/or normalisation such as vector normalisation and/or baseline correction, or combination thereof.

10. The method according to claim 9 wherein the, or each, pre-processed spectra is then further processed to provide one or more dimensionally reduced spectrum.

11. The method according to claim 10 wherein spectra is processed to provide second derivative spectra with respect to wavenumber (or frequency).

12. A method for monitoring the progression of lung cancer in a human subject comprising repeating the method of claim 1, and ideally the same, method(s) periodically.

13. A method for treating lung cancer, comprising performing the method of claim 1, and depending upon the outcome of the method, where cancer is indicated undertaking a suitable or selected course of treatment.

14. A kit for use in determining lung cancer in a sputum sample from human subject with a high risk or likelihood of developing lung cancer, said kit comprising:
a) an FTIR machine for performing spectral analysis of a sputum sample obtained from the subject;
b) a processing unit, wherein said processing unit processes the spectrum from the sample according to the methods according to claim 1; and
c) an output unit arranged to provide an output indicative of a subject suffering from lung cancer in the subject according to a determination made by the processing unit in step b).

15. The kit according to claim 14 further comprising a collection means for collecting the sputum samples.

16. The kit according to claim 15 wherein said collection means is provided as an infrared transparent slide.

17. The kit according to claim 16 wherein said kit comprises a sample or slide holder.
